(19) United States
(12) Patent Application Publication
(10) Pub. No.: US 2022/0395223 A1
(43) Pub. Date: Dec. 15, 2022

(54) DEVICE FOR MEASURING, PROCESSING AND TRANSMITTING IMPLANT PARAMETERS

(71) Applicant: AO Technology AG, Chur (CH)

(72) Inventor: Markus WINDOLF, Davos (CH)

(21) Appl. No.: 17/770,596

(22) PCT Filed: Oct. 16, 2020
(86) PCT No.: PCT/CH2020/000015

§ 371 Date: Apr. 20, 2022

(30) Foreign Application Priority Data
Oct. 22, 2019 (CH) 01335/19

(51) Int. Cl. A61B 5/00

(52) U.S. Cl. CPC A61B 5/4851; A61B 5/6847; A61B 5/0031; A61B 5/4504; A61B 5/0022; A61B 2562/0261; A61B 2562/16; A61B 2560/0214; A61B 2560/0406

(57) ABSTRACT

A device (1) for measuring, processing and transmitting implant parameters in osteosynthesis, the device (1) comprising: a biocompatible sterilizable housing (2); a strain sensor (3); an electronic unit (4) to process electrical signals provided by the strain sensor (3), wherein the housing (2) comprises (i) a measurement portion (5) of the height H5 comprising a cavity (51); and (ii) a compartment portion (6) of the height H6 with a cavity (61), and wherein the measurement portion (5) comprises at least two affixing means (7) for affixing the device (1) to an implant and wherein the electronic unit (4) is positioned in the cavity of the compartment portion (6).

### BACKGROUND OF THE INVENTION

### 1. Field of the Invention

The invention relates to a device for measuring, processing and transmitting implant parameters in osteosynthesis according to the preamble of claim 1, an assembly according to the preamble of claim 48 and to a method for monitoring and/or controlling a medical implant according to the preamble of claim 50.

### 2. Description of the Related Art

A device for measuring, processing and transmitting implant parameters in osteosynthesis is known from US 2016/0128573 A1. This known device comprises a bridge provided with one or more strain gauges and having to two opposing ends. Each a clamp is coupled to one end of the bridge so that the bridge can be affixed via the clamps to a spinal fusion rod implanted parallel to patient's spine. A separate housing which encapsulates the control circuitry is attached to the bridge. Strain occurring on the spinal fusion rod is mechanically transferred from the rod via the clamps to the bridge. Strain sensed by the strain gauges is transformed into electric signals by means of the strain gauges. These electric signals are received by the control circuitry which is electrically coupled to the strain gauges by means of pins. The control circuitry is configured to convert the electric signals received from the strain gauges into digital data and to wirelessly transmit that digital data to a remote computing device. A drawback of this known device is that the strain sensor is positioned with a considerable spacing over the implant so that the bridge may be subjected to strain resulting from other forces that may be exerted on the device by nearby muscles or other adjacent tissue. Additionally, the surrounding soft tissue is irritated by this voluminous device.

### BRIEF SUMMARY OF THE INVENTION

It is an object of the invention to provide a device for measuring, processing and transmitting implant parameters in osteosynthesis which permits to minimize irritation of surrounding anatomical structures and soft tissue and which reduces interferences induced by forces of nearby muscles and/or other adjacent tissue.

The invention solves the posed problem with a device for measuring, processing and transmitting implant parameters in osteosynthesis comprising the features of claim 1, an assembly comprising the features of claim 47 and with a method for monitoring and/or controlling a medical implant comprising the features of claim 48.

The advantages of the device according to the invention can be seen therein that due to the inventive device:
irritation of soft tissue surrounding the device can be minimized due to the fact that the measurement portion with the strain sensor can be positioned on top of the implant but the complete electronic unit including bulky components (e.g. batteries) can be positioned laterally from the implant in a cavity of the compartment portion;
interferences induced by forces of nearby muscles and/or other adjacent tissue can thereby be significantly reduced; and
transferred strain to be measured by a strain sensor can be maximized by relocating other structures and elements out of the strain transition path.

Further advantageous embodiments of the invention can be commented as follows:

In a special embodiment the measurement portion comprises at least two affixing means for releasably affixing the device to an implant.

In a further special embodiment, the height H5 of the measurement portion is smaller than the height H6 of the compartment portion, the relation H5:H6 being preferably smaller than 0.5.

In another embodiment the maximum height of the measurement portion does not exceed 4 mm, preferably 2 mm. Bulky components such as batteries can be placed at the periphery, preferable beside the implant to avoid extensive device protrusion above the implant and hence irritation of anatomical structures and soft tissues.

In another embodiment the measurement portion is provided with a vertical throughgoing slot adjacent to the compartment portion, wherein the measurement portion has a length L5 measured along a line connecting the centers of the affixing means and the slot preferably extends over 30 to 90% of the length L5 of the measurement portion. The slot permits to transmit stresses and strains to the strain sensor instead of transmitting them to the compartment portion. Only minimal mechanical strain is transferred to the compartment portion reducing the risk of compartment or electronics failure.

In another embodiment the compartment portion has a length L6 measured along a line connecting the centers of the affixing means and the measurement portion is provided with a vertical throughgoing slot adjacent to the compartment portion, wherein the length L6 is smaller than the length L5 and the slot preferably extends over 30 to 90% of the length L6 of the compartment portion.

In another embodiment the upper surface of the measurement portion and a top surface of the compartment portion form an upper free surface of the device which is planar, preferably in the form of a straight surface. The height of the assembly consisting of the device and the implant can be minimized due to the compartment portion being arranged laterally from the implant.

In a further embodiment the device has an L-shaped cross-sectional area orthogonal to a line connecting the centers of the affixing means, wherein
(a) the upper surface of the measurement portion and the top surface of the compartment portion form an upper free surface of the device;
(b) the cross-sectional area of the measurement portion forms a first leg and the cross-sectional area of the compartment portion extends along the second leg of the L-shaped cross-sectional area; wherein
(c) the cross-sectional area of the measurement portion extends with its height H5 from the upper free surface of the device measured parallel to the second leg and the cross-sectional area of the compartment portion extends with its height H6 from the upper free surface of the device in the direction of the second leg and protrudes beyond the lower surface of the measurement portion, so that the lower surface of the measurement portion is positionable on a top surface of a bone plate while the compartment portion extends beside the bone plate.

By these means bulky components can be placed at the periphery, preferable beside the implant to avoid extensive device protrusion above the implant and hence irritation of anatomical structures and soft tissues. The affixing means can be positioned closer to each other (only the strain sensor positioned in between) to account for various screw hole patterns on existing implants. Sensitive electronics are positioned out of the force transmission path to avoid mechanically induced failure.

In a further embodiment the measurement portion comprises a strain concentration area located in between the at least two affixing means, wherein the strain sensor is configured to measure strain at the strain concentration area. The advantage of this configuration is that the affixing means permit to transmit mechanical load to the measurement portion.

In a further embodiment the compartment portion is mechanically connected to the measurement portion by means of the connection portion. Therewith the advantage can be achieved that only minimal mechanical strain is transferred to the compartment portion reducing the risk of compartment or electronics failure. At the same time strain measurement sensitivity is increased since the bulk of the strain is concentrated at the strain concentration area. This can be realized by a longitudinal slot.

Preferably, the connection portion is remote to the strain concentration area.

In another embodiment the compartment portion and the measurement portion are integral with the connecting portion.

In a further embodiment the connecting portion is mountable to the compartment portion and to the measurement portion.

In a further embodiment the cavity of the measurement portion and the cavity of the compartment portion are sealed by a cover so that the strain sensor and the electronic unit are is located in a sealed cavity.

Preferably, the sensor cavity and the compartment portion form a hermetic or near-hermetic seal to the environment.

In another embodiment the housing has—along a line connecting the centers of the affixing means—an oblong shape with a first end, a second end and a length L, wherein the compartment portion is mechanically connected to the measurement portion by means of the connection portion over the full length of the shorter of the measurement portion and the compartment portion.

In another embodiment the connecting portion comprises a second slot.

In again another embodiment the connecting portion comprises at least one or more slots reducing the cross-sectional area of the connection portion between the measurement portion and the compartment portion maximum to 50%, preferably maximum to 40% of the whole length of the shorter of the measurement portion and the compartment portion.

In a further embodiment the strain concentration area is provided by the cavity of the measurement portion to accommodate the at least one strain sensor.

In a further embodiment the strain concentration area comprises a recess in the contact surface of the measurement portion opposite the cavity of the measurement portion to reduce local transverse cross sectional area in relation to the transverse cross sectional area of the measurement portion. This additional recess or groove at the contact surface underneath the measurement portion permits to concentrate strain.

In yet a further embodiment the affixing means are configured as through holes for receiving fasteners.

Preferably, the affixing means are configured as a plurality of through holes to accommodate various existing hole patters of available implants.

In another embodiment the undersurface of the affixing means is designed to abut with a circular flat surface. Specially designed inserts will lock in the angular stable locking holes of the bone plate and will minimally protrude out of the bone plate to establish contact to the device's undersurface (bridge configuration to transmit load).

In another embodiment the affixing means comprise at least one fastener or at least one clamp.

In a further embodiment the electronic unit comprises an electronic data processing device electrically connectable or connected to the strain sensor, a data memory electrically connected to the data processing device and suitable to store data received from the data processing device, a data transmission device electrically connected to the data memory, and a power supply.

Preferably, the device comprises an antenna for wireless data transmission, which is recessed in a pocket in the housing.

In another embodiment at least one strain sensor is attached to the inner wall of the cavity of the measurement portion, which is closest to the contact surface. This configuration permits the advantage that the sensor is shielded from parasitic strain acting on the housing from e.g. muscle contractions in contact with the housing.

In a further embodiment the housing is made of a biocompatible but non-biodegradable metallic or polymeric material, preferably Titanium or Titanium alloys, Stainless Steel, Polyetheretherketone (PEEK) or Liquid Crystal Polymer (LCP).

In a further embodiment the electronic unit additionally comprises an accelerometer-based event detector configured to control sleep- and wake-up stages of the electronic unit based on body movement. The system can be in sleep mode unless the patient activates it via body movement in order to save energy when no measurement is required.

In another embodiment the electronic data processing device comprises at least one peak-valley detector to extract extreme values corresponding to signal amplitudes from the measured signal.

Preferably, the at least one peak-valley detector extracts signal amplitudes in real-time. In another embodiment the at least one peak-valley detector is programmed to detect and supply signal amplitudes above a predefined amplitude threshold and is programmed to count the detected amplitudes above said amplitude threshold.

In another embodiment the at least one peak-valley detector is programmed to detect and supply the elapsed time between detected signal amplitudes above a predefined amplitude threshold defined as event-pause.

In a further embodiment the electronic data processing device is programmed to calculate statistically relevant data based on measurement data received from the one or more sensor(s) and to store the statistical data in the data memory. This configuration permits the advantage to reduce the amount of data to be stored in the data memory and to be wirelessly transferred, in order to save energy, in order to minimize implant volume and maximize device lifetime.

Preferably, the electronic data processing device is programmed to calculate statistically relevant data for a defined and recurring time period. Currently evaluation periods are 6 h, 24 h. Offline (not on the implant) also a 1-week moving average calculation is possible.

In a further embodiment the data processing device is programmed for continuous data collection accumulating to in average at least 1 h collection time per day, preferable 24 h per day. So, the advantage of disrupted collection and energy saving in sleep mode is achievable. The data collection can be fragmented such as 1 h daily or 6 times 10 min per day, or 7 h once a week etc.

In a further embodiment the electronic data processing device is programmed to calculate statistically relevant data from signal amplitude values and counts obtained from the at least one peak-valley detector.

Preferably, the statistically relevant values are selected from the list, but not limited to,
Arithmetic mean of amplitudes or event-pauses
Standard deviation of amplitudes or event pauses
Minimum and maximum amplitude or event pause
Median and percentiles of amplitudes or event-pauses
Histogram of amplitudes or event-pauses
Total counts of amplitudes or event-pauses

In a further embodiment the statistical values are calculated for the valley strain as obtained from the at least one peak-valley detector. This configuration permits the advantage that valley strain remains constant if deformation happens in the linear elastic range. Change in valley strain might indicate plastic deformation of the implant and can hence be used to detect early onset of implant failure.

In another embodiment statistically relevant values are calculated for a defined number of largest detected amplitudes. This approach permits focusing on high magnitude amplitudes and eliminating the influence of parasitic low magnitude amplitudes negatively influencing the results.

In another embodiment the data transmission device is configured as a wireless data transmitter based on a wireless technology standard, preferably Bluetooth, RFID, NFC or ZigBee.

In another embodiment the power supply is a primary or rechargeable battery, a capacitor or a fuel cell.

In again another embodiment the rechargeable battery or capacitor is chargeable by energy induction or by energy harvesting, for example by deriving thermal energy from a patient's body, kinetic energy from body movements, deformation energy from the implant under functional loading or by harvesting energy from surrounding electromagnetic fields.

In a further embodiment at least one or more sensor(s) is/are suitable to obtain measurement data related to at least one of the following physical quantities: load applied to an implant, strain in an implant and relative displacement of implant parts.

In a further embodiment the one or more sensor(s) is/are selected from the following group of measuring probes: inductivity meters, capacitance meters, incremental meters, strain gauges, particularly wire resistance or capacitive strain gauges, load cells, piezo based pressure sensors, accelerometers, gyroscopes, goniometers, magnetometers, humidity sensors, temperature sensors.

In a further embodiment a second or more sensor(s) are placed in the compartment portion.

Preferably, the device is suitable to be affixed to an implant selected from the list, but not limited to:
bone plates;
spinal implants (pedicel screw-rod system);
external fixator struts/rods;
Schanz screws or Steinman pins; or
rods.

According to a further aspect of the invention an assembly is provided which comprises the device according to the invention, two bone screws, two inserts to contact the lower surface of the measurement portion and a bone plate. The assembly may also comprise a device according to the invention and a bone plate permanently connected to the device, preferably by means of welding.

According to a further aspect of the invention a method for monitoring a medical implant by using the device according to the invention is provided which comprises the following steps: A) Obtaining measurement data by means of the strain sensor; B) Performing real-time processing on the measurement data obtained under step A) and by means the data processing device; C) Calculating statistical data based on the processed data under step B); D) Storing the statistical data in the data memory; E) Inquiring and downloading selected data stored in the data memory by means of an external data receiver; and F) transmitting the downloaded selected data from the external data receiver to a computer for further data management and processing.

In a further embodiment in step D) the statistical data is automatically stored in the data memory at defined time points over the day-cycle or on manual request.

In a further embodiment in step A) the measurement data is continuously collected during a selectable period of time, preferably with a sampling frequency of 9-30 Hz, most preferably of 10-30 Hz.

In another embodiment in step C) the statistical data is calculated by using evaluation intervals of minimum 4 hours, preferably of minimum 6 hours.

In a further embodiment the electronic unit compromises a real-time clock synchronizable to an external clock. By this means evaluation intervals can be allocated to specific times in the patient's day cycle.

In another embodiment in step C) the statistical data is calculated by using evaluation intervals of maximum 48 hours, preferably of maximum 24 hours.

In another embodiment in step C) the statistical data is automatically calculated in selectable evaluation intervals.

In again another embodiment in step E) the term for inquiring and downloading selected data is freely selectable by a user.

In a further embodiment step E) is automatically performed at defined time points by the device and the external data receiver without the need for user interaction.

In a further embodiment the external data receiver is a smartphone suitably programmed to inquire and download data from the device.

In a further embodiment the external computer is configured as a webserver with a database for data collection.

In a further embodiment the method comprises a calibration procedure before step A), wherein the calibration procedure comprises the steps of: i) Positioning a patient on a scale or on a force-plate; ii) Recording the load applied on the scale or on the force-plate or limb loading device; iii) Reading out the actual output of the strain sensor; iv) Repeating steps ii and iii with a load applied different from the first load; v) Calculating a linear calibration factor using the difference in loads recorded under step ii) and the difference in actual outputs of the strain sensor read out under step iii); and vi) Calculating a linear calibration factor using the load recorded under step ii) and the actual output of the strain sensor read out under step iii); and vii) Storing the linear calibration factor.

Preferably, the device according to the invention is used for:
monitoring of bone healing in osteosynthesis;
for monitoring a bone distraction implant;
monitoring spinal fusion progress.

Preferably, the external data receiver comprises a wireless internet connection.

### A BRIEF DESCRIPTION OF THE DRAWINGS

Several embodiments of the invention will be described in the following by way of example and with reference to the accompanying drawings in which:

FIG. 1 illustrates a perspective view of an embodiment of the device according to the invention;

FIG. 2 illustrates a sectional view of the housing of FIG. 1 orthogonal to the force transmission path;

FIG. 3 illustrates a perspective view of another embodiment of the device according to the invention with the cavity of the measurement portion hermetically sealed;

FIG. 4 illustrates a sectional view of the housing of a further embodiment of the device according to the invention along the force transmission path;

FIG. 5 illustrates a magnified view of detail A in FIG. 4;

FIG. 6 illustrates a perspective view of a further embodiment of the device according to the invention;

FIG. 7 illustrates a lateral view of the embodiment of FIG. 6; and

FIG. 8 illustrates a top view of the embodiment of FIG. 6.

### DETAILED DESCRIPTION OF THE INVENTION

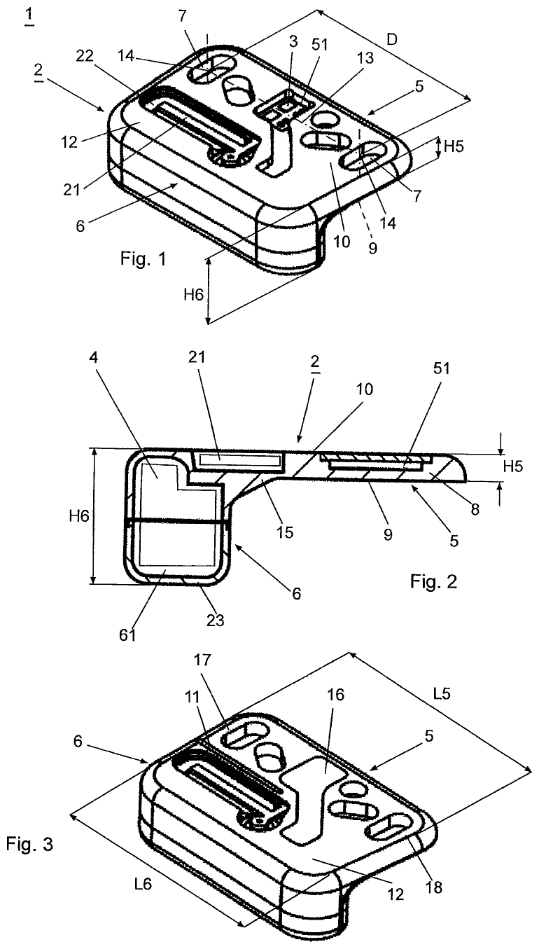

FIGS. 1 and 2 illustrate an embodiment of the device 1 for measuring, processing and transmitting implant parameters in osteosynthesis according to the invention, wherein the device 1 essentially comprises a biocompatible sterilizable housing 2 which is partitioned into a measurement portion 5 of the height H5 and comprising a cavity 51 and a compartment portion 6 of the height H6 with a cavity 61, a strain sensor 3 arranged in the cavity 51 of the measurement portion 5, an electronic unit 4 to process electrical signals provided by the strain sensor 3, wherein the electronic unit 4 is positioned in the cavity 61 of the compartment portion 6. The measurement portion 5 comprises a plurality of affixing means 7 for affixing the device 1 to an implant. The cavity 51 of the measurement portion 5 is electrically connected to the cavity 61 of the compartment portion 6 to transmit electrical signals provided by the strain sensor 3 to the electronic unit 4 for further processing. The measurement portion 5 comprises a lower contact surface 9 configured to contact a top surface of a bone plate (not shown) and spaced apart therefrom by the height H5 an upper surface 10. Exemplarily, but not limiting, the relation H5:H6 is about 0.25 and the maximum height of the device 1 above the contact surface 9 is about 2 mm.

In alternative embodiments the relation H5:H6 may be greater than 0.25 but smaller than 0.5, preferably smaller than 0.35 so that the maximum height of the device 1 above the contact surface 9 does not exceed 4 mm, preferably 2 mm.

The strain sensor 3 is positioned in the cavity 51 of the measurement portion 5 in the proximity of the contact surface 9. The electronic unit 4 comprises an electronic data processing device electrically connectable or connected to the strain sensor 3, a data memory electrically connected to the data processing device and suitable to store data received from the data processing device, a data transmission device electrically connected to the data memory and a power supply. In alternative embodiments the device 1 may additionally comprise one or more additional sensors, an electronic signal conditioner and an analog-digital converter. Further, the device 1 comprises an antenna 21 for wireless data transmission, which is recessed in a pocket 22 in the housing 2.

The device 1 has an L-shape in a cross-section orthogonal to a line 13 connecting the centers 14 of the affixing means 7 so that the measurement portion 5 is positionable on a top surface of a bone plate while the compartment portion extends beside the bone plate. The upper surface 10 of the measurement portion 5 and a top surface 12 of the compartment portion 6 form an upper free surface of the device 1 which is planar and in the form of a straight surface. The measurement portion 5 comprises a strain concentration 8 area located in between the at least two affixing means 7, wherein the strain sensor 3 is configured to measure strain at the strain concentration area 8. The strain concentration area 8 is meant to be a region with reduced cross sectional area (transverse section) of the measurement portion 5 in order to maximize the local material strain under a given load running over the measurement portion 5. It can be realized by a cavity or a transverse groove from either side of the measurement portion 5. In the present embodiment the strain concentration area 8 includes the cavity 51 of the measurement portion 5 to accommodate the at least one strain sensor 3. The strain sensor 3 is attached to the inner wall of the cavity 51 of the measurement portion 5 which closest to the contact surface 9.

The compartment portion 6 is mechanically connected to the measurement portion 5 by means of the connection portion 15. This connection portion 15 is remote to the strain concentration area 8. Exemplarily, but not limiting, the compartment portion 6 and the measurement portion 5 are integral with the connecting portion 15. In alternative embodiments the connecting portion 15 is mountable to the compartment portion 6 and to the measurement portion 5.

The affixing means 7 are configured as through holes for receiving fasteners. Exemplarily, the affixing means 7 are configured as a plurality of through holes to accommodate various existing hole patters of available implants. Along the line 13 connecting the centers 14 of the affixing means 7 the housing 2 has an oblong shape with a first end 17, a second end 18 and a length L. The line 13 connecting the centers 14 of the affixing means 7 also defines the force transmission path, along which stresses induced by these forces occur. The compartment portion 6 is mechanically connected to the measurement portion 5 by means of the connection portion 15 over the full length L.

The compartment portion 6 comprises a cap 23 to close the cavity 61 of the compartment portion 6. The cavity 51 of the measurement portion 5 is sealed by a cover 16 so that the strain sensor 3 is located in a sealed cavity so that the sensor cavity and the compartment portion 6 form a hermetic or near-hermetic seal to the environment.

The housing 2 is made of a biocompatible but non-biodegradable metallic or polymeric material, preferably Titanium or Titanium alloys, Stainless Steel, Polyetheretherketone (PEEK) or Liquid Crystal Polymer (LCP). The power supply is a primary or rechargeable battery, a capacitor or a fuel cell, wherein the rechargeable battery or capacitor is chargeable by induction or by energy harvesting, by deriving thermal energy from a patient's body, kinetic energy from body movements, deformation energy from the implant under functional loading or by harvesting energy from surrounding electromagnetic fields.

In alternative embodiments the device 1 may additionally comprise further strain sensors 3 arranged in the measurement portion 5 and/or a second or more sensor(s) which are placed in the compartment portion 6.

A further embodiment of the device 1 according to the invention is illustrated in FIG. 3 which differs from the embodiment of FIGS. 1 and 2 only therein that the measurement portion 5 is provided with a vertical throughgoing slot 11 adjacent to the compartment portion 6. Exemplarily, but not limiting, the slot 11 extends over 50% of the length L5 of the measurement portion 5. In alternative embodiments the slot 11 may extend between 30 to 60% of the length L5 of the measurement portion 5. Due to the slot 11 stresses and strains are transmitted to the strain sensor 3 but not to the compartment portion 6.

In alternative embodiments the length L5 of the measurement portion 5 and the length L6 of the compartment portion 6 may be different and the slot 11 may extend over 30 to 60% of the shorter of the length L5 of the measurement portion 5 and the length L6 of the compartment portion. The connecting portion 15 may comprises more than slot 11 to reduce the cross-sectional area of the connection portion 15 between the measurement portion 5 and the compartment portion 6 maximum to 70%, preferably maximum to 60% of the shorter of the length L5 of the measurement portion 5 and the length L6 of the compartment portion 6. In other embodiments the one or more slots 11 may reduce the cross-sectional area of the connection portion 15 between the measurement portion 5 and the compartment portion 6 maximum to 50%, preferably maximum to 40% of the shorter of the length L5 of the measurement portion 5 and the length L6 of the compartment portion 6.

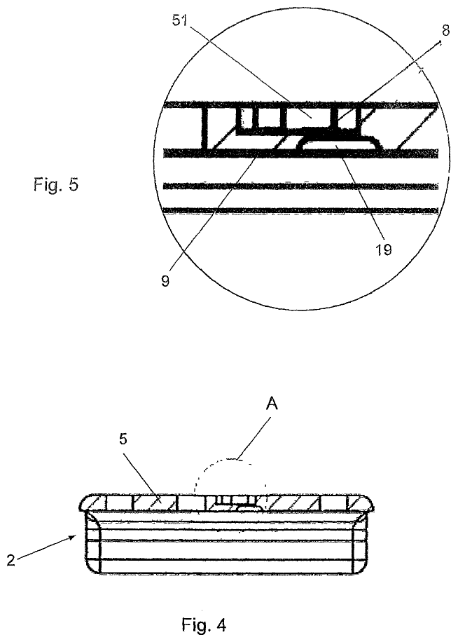

FIGS. 4 and 5 illustrates another embodiment which differs from the embodiment of FIGS. 1-3 only therein, that the strain concentration area 8 comprises a recess 19 in the contact surface 9 of the measurement portion 5 opposite the cavity 51 of the measurement portion 5 to reduce local transverse cross sectional area in relation to the transverse cross sectional area of the measurement portion 5.

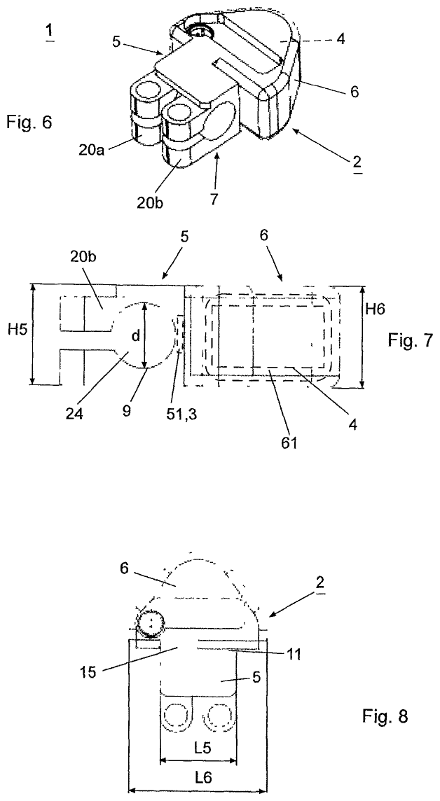

A further embodiment of the device 1 according to the invention is illustrated in FIGS. 6-8 wherein the device 1 comprises:

A) a biocompatible sterilizable housing 2;
B) a strain sensor 3;
C) an electronic unit 4 electrically connected to the strain sensor 3 and configured to process electrical signals provided by the strain sensor 3, wherein
D) the housing 2 comprises
(i) a measurement portion 5 of the height H5 and comprising a cavity 51; and
(ii) a compartment portion 6 of the height H6 with a cavity 61, and wherein
E) the measurement portion 5 comprises:
at least two affixing means 7 for affixing the device 1 to an implant, wherein the at least two affixing means 7 are spaced apart from each other, wherein
the cavity 51 of the measurement portion 5 is arranged between the at least two affixing means 7; and wherein
the strain sensor 3 is positioned in the cavity 51 of the measurement portion 5; and wherein
F) the electronic unit 4 is positioned in the cavity 61 of the compartment portion 6.

The configuration of the embodiment of FIGS. 6-8 differs from the embodiments of FIGS. 1-5 only therein that the height H5 of the measurement portion 5 is exemplarily, but not limiting, essentially equal to the height H6 of the compartment portion 6. In alternative embodiments the height H5 of the measurement portion 5 may be different from the height H6 of the compartment portion 6. Furthermore, the affixing means 7 comprise two clamps 20a,20b to attach the device to a longitudinal rod, e.g. a spinal rod of a spinal fusion device. Exemplarily, the clamps 20a,20b are integral with the measurement portion 5. Each clamp 20a,20b includes a curved contact surface 9 forming a channel 24 with the shape of a portion of a circular cylinder with the diameter d so that a longitudinal rod is positionable in the channels 24 of both clamps 20a,20b. Thereby, the contact surface 9 is located at a lateral portion of the measurement portion 5 which is remote from the compartment portion 6.

Additionally, in alternative embodiments the device 1 according to the invention may comprise one or more of the following features:
the electronic unit 4 additionally comprises an accelerometer-based event detector configured to control sleep- and wake-up stages of the electronic unit based on body movement;
the electronic data processing device is electrically connectable to at least one sensor 3 through a signal conditioner and analog-digital converter allowing to process measured signals received from said at least one sensor;
the data memory is electrically connected to said signal processing device allowing to store data received from said signal processing device;
the data transmission device is electrically connected to said data memory for transmitting data received from said data memory to a remote data receiving device which is connectable to an external data processing device;
the electronic data processing device comprises at least one peak-valley detector to extract extreme values corresponding to signal amplitudes from the measured signal, wherein the at least one peak-valley detector preferably extracts signal amplitudes in real-time;
the at least one peak-valley detector is programmed to detect and supply signal amplitudes above a predefined amplitude threshold; and is programmed to count the detected amplitudes above said amplitude threshold;
the at least one peak-valley detector is programmed to detect and supply the elapsed time between detected signal amplitudes above a predefined amplitude threshold defined as event-pause;
the electronic data processing device is programmed to calculate statistically relevant data based on measurement data received from the one or more sensor(s) and to store the statistical data in the data memory;
the electronic data processing device is programmed to calculate statistically relevant data for a defined and recurring time period;
the data processing device is programmed for continuous data collection. the electronic data processing device is programmed to calculate statistically relevant data from signal amplitude values and counts obtained from the at least one peak-valley detector;
statistically relevant values are selected from the following list, but not limited to,
   Arithmetic mean of amplitudes or event-pauses
   Standard deviation of amplitudes or event pauses
   Minimum and maximum amplitude or event pause
   Median and percentiles of amplitudes or event-pauses
   Histogram of amplitudes or event-pauses
   Total counts of amplitudes or event-pauses
statistically relevant values are calculated for a defined number of largest detected amplitudes
the data transmission device is configured as a wireless data transmitter based on a wireless technology standard, preferably Bluetooth, RFID, NFC or ZigBee;
at least one of the one or more sensor(s) is suitable to obtain measurement data related to at least one of the following physical quantities: load applied to an implant, strain in an implant and relative displacement of implant parts; and
the one or more sensor(s) is/are selected from the following group of measuring probes: inductivity meters, capacitance meters, incremental meters, strain gauges, particularly wire resistance or capacitive strain gauges, load cells, piezo based pressure sensors, accelerometers, gyroscopes, goniometers, magnetometers, humidity sensors, temperature sensors.

A preferred embodiment of the method for monitoring and/or controlling an implant essentially comprises the following steps: A) obtaining measurement data by means of the strain sensor 3; B) performing real-time processing on the measurement data obtained under step A) by e.g. employing one or several real-time min-max detectors with different sensitivity thresholds and respective peak counters; C) calculating statistical parameters, such as the sum of maxima and minima and the peak counts in real-time based on the processed data under step B); D) automatically storing the statistical parameters in the data memory at defined time points over the day cycle or on manual request; E) inquiring and downloading selected data stored in the data memory by means of an external data receiver; and F) transmitting the downloaded selected data from the external data receiver to an external computer for further data management and processing. The patient data can be exemplarily but not limiting recorded and analyzed in the central computer to efficiently produce statistical reference plots to improve the interpretation of the data. If a determination of the patient's activity is of interest, e.g. the number of steps per hour and the intensity distribution of the steps an activity histogram can be generated on the basis of the continuously recorded data. By this means a topical feedback related to the strain of the fracture can be obtained for the doctor and the patient so as to permit an active exerting of influence for the patient. For this reason, in step A) the measurement data is preferably continuously collected during a selectable period of time, preferably with a sampling frequency of 9-30 Hz, most preferably of 10-30 Hz.

Due to a selected evaluation interval between 4 hours and 24 hours for calculating the required statistical data by means of the data processing device the data to be transmitted via the data transmission device to an external data receiver can be significantly reduced. By this means, the energy demand for data transmission can be reduced which usually is the major part of the energy consumption of the data acquisition device so that an autonomous operation of the device 1 during at least four months can be achieved.

The patient can inquire and download data at any time or even withdraw from inquiring data for several weeks without losing data. The external data receiver may be a smartphone suitably programmed to inquire and download data from the device 1. The inquiry of data may be performed passively, e.g. via an automatic link acquisition of the smartphone once a week so as to permit the patient to be independent of the clinic. Therefore, in step E) the term for inquiring and downloading selected data is freely selectable by a user.

Exemplarily but not limiting an external data processing can be performed as follows: The data may be either downloaded and stored on the external computer or directly processed in the data receiving device, e.g. a smartphone. The sensor response is calibrated to physical units using a linear approach by utilizing a predefined or patient specific scale factor. A statistical relevant value will be selected for data processing, e.g. the arithmetic mean of amplitudes. if no calibration of the data was performed, the data accumulated over the elapsed recording time can be normalized to the maximum occurred value over time. Data will be plotted over time and provided to the user for therapeutic decision making. A decline in the curve indicates reduction in elastic deformation of the osteosynthesis through increased load sharing of the stiffening bone during healing, whereas no significant change of the curve indicates absence of healing or pathological bone healing. Based on the shape of the curve the user may decide for timely operational or non-invasive intervention or may steer physiotherapy for early regain of patient activity and weight bearing, or to accelerate the healing progression, or to avoid mechanical failure of the osteosynthesis.

The evaluation interval length determines scattering of the data from natural variances in functional loading of the patient. Longer evaluation intervals lead to reduced scattering. Hence, it can be beneficial to increase evaluation interval length during post processing by averaging several evaluation intervals or by applying filtering such as moving-average filtering.

### Meaning of the Results and Presentation

The mentioned evaluations may be visualized by plotting the measured and processed values over time in absolute or relative terms (normalizing the sensor response to the initial postoperative response of the sensor). For instance, the healing process may be visualized with decreasing average amplitude from peak-valley detection over time. A threshold can be set for determining the optimal time point for implant removal. Mal-unions may be identified at an early stage and different dynamization protocols can be evaluated. The amplitude histogram or percentiles gives information about the patient's activity over time and therefore about the stimulation of the bone. For monitoring distraction implants or segment transport implants or segment transport implants, the current sensor value provides valuable information about the progression of the distraction process.

### Application Examples of the Medical Device According to the Invention

1) Monitoring of bone healing in osteosynthesis following the principle of secondary healing. The strain in a standard bone plate or intramedullary nail or external fixator rod measured by strain gauges could be acquired and processed with the device 1. Reduction of strain could be interpreted as enhanced load sharing of the bone and as progress in the bone consolidation. Knowledge about the healing progression is valuable information to detect non-unions at an early stage or to determine an optimal time-point for implant removal.

Mechanical stimulation of bone is known to promote bone formation. A tool to monitor dynamization of newly proposed dynamic implants and its progression over time is also an application field for the device 1. It offers the opportunity to acquire long term and continuous data rather than repeated short term measurements as done by known techniques.

2) Monitoring of a distraction or segment transport implant. The method of distracting bone is used for generation of new bone tissue for critical size defects or bone lengthening. The distraction and bone consolidation process can be monitored by measuring the strain in e.g. the struts or Schanz pins of an external fixation construct used for bone segment transport, e.g. a Taylor Spatial Frame,

3) Monitoring of implant failure. Catastrophic failure of orthopedic implants from physiological overloading is a common devastating problem leading to re-operation. Data delivered by the device according to the invention may be used for detection of early onset of implant failure. E.g. change in average valley strain over time might indicate onset of plastic implant deformation which might climax in catastrophic failure. Physiotherapy and weight bearing recommendations can be adjusted accordingly.

4) Monitoring of spinal fusion. Fusing two or more vertebral body segments is a common orthopedic procedure in spinal surgery. Objective knowledge of the bony fusion status is important for therapeutic decision making. By measuring the spinal rod deformation, this process can be monitored. The drawback of known solutions in the field is their snap-shot nature, where measurements are only performed at distinct time points. Complex physiological loading at the spine makes interpretation of such isolated short-term measurements difficult. Data remains inconclusive. In contrast, the proposed invention utilizes continuous data collection with statistical evaluation, reducing the influencing of functional loading variances and thereby rendering the acquired data relevant.

In spine the device according to the invention may further be used for controlling deformity corrections such as scoliosis and early detection of implant failure.

Additional or alternative application examples may be:
Measurement of blood sugar and counteraction by controlled release of Insulin. Blood sugar values are monitored and processed over a certain time period and used for controlling deliverance of medication. This can be realized as autonomous control loop inside the body. The values have to be transferred to an external receiver to control the process.
Arterial blood gas monitoring (O₂, CO₂, blood pressure).
Lactate concentrations.

Although the invention has been described in conjunction with specific embodiments thereof, it is evident that many alternatives, modifications and variations will be apparent to those skilled in the art. Accordingly, it is intended to embrace all such alternatives, modifications and variations that fall within the scope of the appended claims.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

1-70. (canceled)

71: A device for measuring, processing and transmitting implant parameters in osteosynthesis, the device comprising:
a biocompatible sterilizable housing;
a strain sensor; and
an electronic unit electrically connected to the strain sensor and configured to process electrical signals provided by the strain sensor;
wherein the housing comprises
a measurement portion having a height H5, an upper surface, a lower contact surface and a measurement portion cavity, and
a compartment portion having a height H6 with a compartment portion cavity,
wherein the measurement portion comprises at least two affixing means for affixing the device to an implant,
wherein the at least two affixing means are spaced apart from each other by a distance D,
wherein the cavity of the measurement portion is arranged between the at least two affixing means,
wherein the strain sensor is positioned in the measurement portion cavity, and
wherein the electronic unit is positioned in the compartment portion cavity.

72: The device according to claim 71, wherein the at least two affixing means are configured for releasably affixing the device to the implant.

73: The device according to claim 71, wherein the height H5 of the measurement portion is smaller than the height H6 of the compartment portion.

74: The device according to claim 71, wherein the height H5 of the measurement portion does not exceed 4 mm.

75: The device according to claim 71, wherein the measurement portion is provided with a slot adjacent to the compartment portion, wherein the measurement portion has a length L5 measured along a line connecting centers of the at least two affixing means, and wherein the slot extends along 30% to 90% of the length L5 of the measurement portion.

76: The device according to claim 71, wherein the compartment portion has a top surface, wherein the upper surface of the measurement portion and the top surface of the compartment portion form a planar, upper free surface of the device.

77: The device according to claim 71, wherein the implant to which the device is affixable is a bone plate, wherein device has an L-shaped cross-sectional area orthogonal to a line connecting centers of the at least two affixing means, wherein the upper surface of the measurement portion and a top surface of the compartment portion form an upper free surface of the device wherein the measurement portion lies within a first leg of the L-shaped cross-sectional area of the device and the compartment portion lies within a second leg of the L-shaped cross-sectional area of the device, wherein the height H5 extends from the upper free surface of the device measured parallel to the second leg and the height H6 extends from the upper free surface of the device through the second leg and protrudes beyond the lower surface of the measurement portion such that the lower surface of the measurement portion is positionable on a top surface of the bone plate while the compartment portion extends beside the bone plate.

78: The device according to claim 71, wherein the measurement portion comprises a strain concentration area located in between the at least two affixing means, and wherein the strain sensor is configured to measure strain at the strain concentration area.

79: The device according to claim 71, wherein the compartment portion is mechanically connected to the measurement portion by means of a connection portion.

80: The device according to claim 71, wherein the strain sensor is sealed within the measurement portion cavity by a cover.

81: The device according to claim 71, wherein the at least two affixing means are through holes in the measurement portion for receiving fasteners.

82: The device according to claim 71, wherein the at least two affixing means are a subset of a plurality of through holes in the measurement portion for accommodating hole patterns of a plurality of different implants.

83: The device according to claim 71, wherein an undersurface of the at least two affixing means is configured to abut with a circular flat surface.

84: The device according to claim 71, wherein the at least two affixing means includes at least one fastener or at least one clamp.

85: The device according to claim 71, wherein the at least two affixing means includes at least one clamp, and wherein the at least one clamp is integral with the measurement portion.

86: The device according to claim 71, wherein the electronic unit comprises an electronic data processing device electrically connectable or connected to the strain sensor, a data memory electrically connected to the data processing device for storing data received from the data processing device, a data transmission device electrically connected to the data memory, and a power supply.

87: The device according to claim 71, wherein the device further comprises an antenna for wireless data transmission, which is recessed in a pocket in the housing.

88: The device according to claim 71, wherein at least one strain sensor is attached to an inner wall of the measurement portion cavity, which is closest to the lower contact surface.

89: The device according to claim 71, wherein the housing is made of a biocompatible non-biodegradable metallic or polymeric material.

90: A method for monitoring a medical implant using a device according to claim 86 affixed to the medical implant, the method comprising the following steps:
A) obtaining measurement data by means of the strain sensor;
B) performing real-time processing on the measurement data obtained in step A) by means of the data processing device;
C) calculating statistical data based on the measurement data as processed in step B);
D) storing the statistical data calculated in step C) in the data memory;
E) inquiring and downloading selected statistical data stored in the data memory in step D) by means of an external data receiver; and
F) transmitting the downloaded selected statistical data from the external data receiver to a computer for further data management and processing.
